(19) United States
(12) Patent Application Publication
(10) Pub. No.: US 2010/0256173 A1
(43) Pub. Date: Oct. 7, 2010

(54) Method of Treating Cognitive Impairment

(76) Inventor: Eckard WEBER, San Diego, CA (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/753,574

(22) Filed: Apr. 2, 2010

Related U.S. Application Data
(60) Provisional application No. 61/166,033, filed on Apr. 2, 2009.

(51) Int. Cl. A61K 31/438; A61P 25/28

(52) U.S. Cl. 514/278

(57) ABSTRACT

This invention relates to the treatment and prevention of cognitive impairment, and particularly to the treatment and prevention of cognitive impairment caused by or associated with Huntington's Disease, Lewy Body Dementia, Pick's Disease and Down's Syndrome.

This application claims priority benefit of U.S. provisional Application No. 61/166,033, filed Apr. 2, 2009, which is hereby incorporated by reference.

### BACKGROUND OF THE INVENTION

1. Field of the Invention

This invention relates to the treatment and prevention of cognitive impairment, and particularly to the treatment and prevention of cognitive impairment caused by or associated with Huntington's Disease, Lewy Body Dementia, Pick's Disease and Down's Syndrome.

2. Background of the Invention

Alzheimer's Disease (AD) is a neurodegenerative disorder for which there are only symptomatic treatments, with limited efficacy. Certain amyloid-beta (Aβ) fragments of the Amyloid Precursor Protein (APP), notably Aβ₁₋₄₀ and Aβ₁₋₄₂ have been implicated in the pathology of AD. Recently, N-terminal fragments of APP (notably sAPP-beta and N-APP) have been postulated to interact with death receptor 6 and activate caspase-dependent axonal pruning and apoptosis of neuronal cells. See Nikolaev et al., Nature 457: 981 (2009).

The compound ST101, also known as spiro(imidazo(1,2-a)pyridin-2(3H)-one-3,2′-indan), is a promising compound for the treatment of AD. ST101 is believed to reduce production of sAPP and thus its activity may be partially due to the ability to reduce activation of the death receptor 6 pathway. There remains a need in the art for therapies that treat and prevent cognitive impairment caused by or associated with Huntington's Disease, Lewy Body Dementia, Pick's Disease and Down's Syndrome.

### BRIEF SUMMARY OF THE INVENTION

The present invention provides a method of treating or preventing cognitive impairment, said method comprising administering a heterocyclic compound having the general Formula (I):

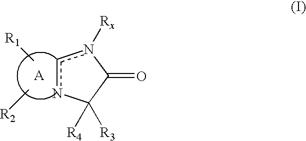

or a pharmaceutically acceptable salt, hydrate or prodrug thereof to a subject in need thereof, wherein the cognitive impairment is caused by or associated with Huntington's Disease, Lewy Body Dementia, Pick's Disease and Down's Syndrome, and wherein another agent for treating or preventing neurodegenerative disease is not administered to the subject.

### DETAILED DESCRIPTION OF THE INVENTION

Unless defined otherwise, all technical and scientific terms used herein have the same meaning as is commonly understood by one of ordinary skill in the art to which this invention belongs.

In one embodiment of the method of the present invention, cognitive impairment is treated. In another embodiment, of the method of the present invention, cognitive impairment is prevented.

In one embodiment of the method of the present invention, the subject has been diagnosed with Huntington's Disease. In another embodiment, the subject has been diagnosed as predisposed to Huntington's Disease. In another embodiment, the subject has been screened to determine whether the subject is predisposed Huntington's Disease.

In one embodiment of the method of the present invention, the subject has been diagnosed with Lewy Body Dementia. In another embodiment, the subject has been diagnosed as predisposed to Lewy Body Dementia. In another embodiment, the subject has been screened to determine whether the subject is predisposed Lewy Body Dementia.

In one embodiment of the method of the present invention, the subject has been diagnosed with Pick's Disease. In another embodiment, the subject has been diagnosed as predisposed to Pick's Disease. In another embodiment, the subject has been screened to determine whether the subject is predisposed Pick's Disease.

In one embodiment of the method of the present invention, the subject has been diagnosed with Down's Syndrome. In another embodiment, the subject has been diagnosed as predisposed to Down's Syndrome. In another embodiment, the subject has been screened to determine whether the subject is predisposed Down's Syndrome.

Although not wishing to be bound by any particular theory, it is believed that ST101 and related heterocyclic compounds of the method of the present invention cause APP to be cleaved to form an approximately 17 kDa fragment that that contains the C-terminal sequence of APP and the amyloid-beta sequence of APP. That is, ST101 and related heterocyclic compounds of the method of the present invention shift APP metabolism from production of harmful APP metabolic products, such as Aβ₁₋₄₂ and Aβ₁₋₄₀, and to production of the approximately 17 kDa fragment. As a result, it is believed that the harmful effects of APP metabolic products such as Aβ₁₋₄₂ and Aβ₁₋₄₀ are mitigated. See U.S. application No. 61/122,689. It is believed that the reduction of Aβ₁₋₄₂ and Aβ₁₋₄₀ and induction of the 17 kDa fragment caused by ST101 are accompanied by a reduction of secreted APP (sAPP-alpha and sAPP-beta). Nikolaev et al., Nature 457: 981 (2009) demonstrated that sAPP-beta and the sAPP-beta derived cleavage product N-APP activate death receptor 6, which mediates axonal pruning and ultimately neuronal loss. Thus, compounds such as ST101 that reduce production of sAPP-beta and/or N-APP are predicted to be neuroprotective. It is known from other studies that ST101 has neuroprotective activity. This activity of ST101 may be, at least in part, due to a reduced activation of the death-receptor 6 pathway.

In one embodiment of the method of the present invention, the subject is a human subject.

The heterocyclic compound of the present invention can be administered at an effective oral dosage of 0.0005 mg per kilogram of body weight or higher. In one embodiment, the compound is administered as part of a unit dosage form containing 5, 10, 15, 20, 25, 30, 35, 40, 45, 50, 55, 60, 65, 70, 75, 80, 85, 90, 95, 100, 120 or 180 mg.

Compositions for use in this invention include all compositions wherein the active ingredient is contained in an amount, which is effective to achieve its intended purpose. While individual needs vary, determination of optimal ranges of effective amounts of each component is within the skill of the art. Typically, the active ingredient may be administered to mammals, e.g. humans, orally at a dose of 0.001 to 3 mg/kg, or an equivalent amount of the pharmaceutically acceptable salt thereof, per day of the body weight of the mammal being treated for AD. The active ingredient may be administered to mammals, e.g. humans, intravenously or intramuscularly at a dose of 0.001 to 3 mg/kg, or an equivalent amount of the pharmaceutically acceptable salt thereof, per day of the body weight of the mammal being treated for AD. Approximately 0.001 to approximately 3 mg/kg can be orally administered to treat or prevent such disorders. If another agent is also administered, it can be administered in an amount, which is effective to achieve its intended purpose.

The unit oral dose may comprise from approximately 0.001 to approximately 200 mg, or approximately 0.5 to approximately 180 mg of the composition of the invention. The unit dose may be administered one or more times daily as one or more tablets, each containing from approximately 0.1 to approximately 90 mg, conveniently approximately 10 to 180 mg of the composition or its solvates. In one embodiment, the unit oral dose can be 10, 20, 30, 40, 50, 60, 70, 80, 90, 100, 110, 120, 130, 140, 150, 160, 170, or 180 mg.

In a topical formulation, the active ingredient may be present at a concentration of approximately 0.01 to 100 mg per gram of carrier.

In addition to administering the active ingredient as a raw chemical, the active ingredient may be administered as part of a pharmaceutical preparation containing suitable pharmaceutically acceptable carriers comprising excipients and auxiliaries, which facilitate processing of the active ingredient into preparations that can be used pharmaceutically. The preparations, particularly those preparations, which can be administered orally, such as tablets, dragees, and capsules, and also preparations, which can be administered rectally, such as suppositories, as well as suitable solutions for administration by injection or orally, can contain from approximately 0.01 to 99 percent, or from approximately 0.25 to 75 percent of active ingredient, together with the excipient.

The heterocyclic compound of Formula (I) can be in the form of hydrate or acid addition salts as a pharmaceutically acceptable salt. Possible acid addition salts include inorganic acid salts such as the hydrochloride, sulfate, hydrobromide, nitrate, and phosphate salts and organic acid salts such as acetate, oxalate, propionate, glycolate, lactate, pyruvate, malonate, succinate, maleate, fumarate, malate, tartrate, citrate, benzoate, cinnamate, methanesulfonate, benzenesulfonate, p-toluenesulfonate, and salicylate salts.

Acid addition salts are formed by mixing a solution of the particular compound of the present invention with a solution of a pharmaceutically acceptable non-toxic acid, such as hydrochloric acid, hydrobromic acid, fumaric acid, maleic acid, succinic acid, acetic acid, citric acid, lactic acid, tartaric acid, carbonic acid, phosphoric acid, sulfuric acid, oxalic acid, and the like. Basic salts are formed by mixing a solution of the particular compound of the present invention with a solution of a pharmaceutically acceptable non-toxic base, such as sodium hydroxide, potassium hydroxide, choline hydroxide, sodium carbonate, Tris, N-methyl-glucamine and the like.

The pharmaceutical compositions of the invention may be administered to any animal, which may experience the beneficial effects of the active ingredient. Foremost among such animals are mammals, e.g., humans and veterinary animals, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner, which is itself known, e.g., by means of conventional mixing, granulating, dragee-making, dissolving, or lyophilizing processes. Thus, pharmaceutical preparations for oral use can be obtained by combining the active ingredient with solid excipients, optionally grinding the resultant mixture and processing the mixture of granules, after adding suitable auxiliaries, if desired or necessary, to obtain tablets or dragee cores.

Suitable excipients are, in particular: fillers, such as saccharides, e.g. lactose or sucrose, mannitol or sorbitol; cellulose preparations and/or calcium phosphates, e.g. tricalcium phosphate or calcium hydrogen phosphate; as well as binders, such as starch paste, using, e.g. maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, e.g. silica, talc, stearic acid or salts thereof, such as magnesium stearate or calcium stearate, and/or polyethylene glycol. Dragee cores are provided with suitable coatings, which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetylcellulose phthalate or hydroxypropymethyl-cellulose phthalate, are used. Dye stuffs or pigments may be added to the tablets or dragee coatings, e.g., for identification or in order to characterize combinations of active ingredient doses.

Other pharmaceutical preparations, which can be used orally, include push-fit capsules made of gelatin, as well as soft, sealed capsules made of gelatin and a plasticizer, such as glycerol or sorbitol. The push-fit capsules can contain the active ingredient in the form of granules, which may be mixed with fillers, such as lactose, binders such as starches, and/or lubricants such as talc or magnesium stearate and, optionally, stabilizers. In soft capsules, the active ingredient can be dissolved or suspended in suitable liquids, such as fatty oils, or liquid paraffin. In addition, stabilizers may be added.

Possible pharmaceutical preparations, which can be used rectally include, e.g. suppositories, which consist of a combination of one or more of the active ingredient with a suppository base. Suitable suppository bases are, e.g. natural or synthetic triglycerides, or paraffin hydrocarbons. In addition, it is also possible to use gelatin rectal capsules, which consist of a combination of the active ingredient with a base. Possible base materials include, e.g. liquid triglycerides, polyethylene glycols, or paraffin hydrocarbons.

Suitable formulations for parenteral administration include aqueous solutions of the active ingredient in water-soluble foam, e.g. water-soluble salts and alkaline solutions. In addition, suspensions of the active ingredient as appropriate oily injection suspensions may be administered. Suitable lipophilic solvents or vehicles include fatty oils, e.g. sesame oil; or synthetic fatty acid esters, e.g. ethyl oleate or triglycerides or polyethylene glycol-400. Aqueous injection suspensions may contain substances, which increase the viscosity of the suspension include, e.g. sodium carboxymethyl cellulose, sorbitol, and/or dextran. Optionally, the suspension may also contain stabilizers.

As used herein, a prodrug is a compound that, upon in vivo administration, is metabolized or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady, Medicinal Chemistry: A Biochemical Approach, Oxford University Press, New York, pages 388 392 (1985)).

Also included within the scope of the present invention are dosage forms of the active ingredient, in which the oral pharmaceutical preparations comprise an enteric coating. The term “enteric coating” is used herein to refer to any coating over an oral pharmaceutical dosage form that inhibits dissolution of the active ingredient in acidic media, but dissolves rapidly in neutral to alkaline media and has good stability to long-term storage. Alternatively, the dosage form having an enteric coating may also comprise a water soluble separating layer between the enteric coating and the core.

The core of the enterically coated dosage form comprises an active ingredient. Optionally, the core also comprises pharmaceutical additives and/or excipients. The separating layer may be a water soluble inert active ingredient or polymer for film coating applications. The separating layer is applied over the core by any conventional coating technique known to one of ordinary skill in the art. Examples of separating layers include, but are not limited to sugars, polyethylene glycol, polyvinylpyrrolidone, polyvinyl alcohol, hydroxypropyl cellulose, polyvinyl acetal diethylaminoacetate and hydroxypropyl methylcellulose. The enteric coating is applied over the separating layer by any conventional coating technique. Examples of enteric coatings include, but are not limited to cellulose acetate phthalate, hydroxypropyl methylcellulose phthalate, polyvinyl acetate phthalate, carboxymethylethylcellulose, copolymers of methacrylic acid and methacrylic acid methyl esters, such as Eudragit®L 12,5 or Eudragit®L 100 (Rohm Pharma), water based dispersions such as Aquateric® (FMC Corporation), Eudragit®L 100-55 (Rohm Pharma) and Coating CE 5142 (BASF), and those containing water soluble plasticizers such as Citroflex® (Pfizer). The final dosage form is either an enteric coated tablet, capsule or pellet.

Examples of prodrugs of the compounds of the invention include the simple esters of carboxylic acid containing compounds (e.g. those obtained by condensation with a C1-4 alcohol according to methods known in the art); esters of hydroxy containing compounds (e.g. those obtained by condensation with a C₁₋₄ carboxylic acid, C₃₋₆ dioic acid or anhydride thereof (e.g. succinic and fumaric anhydrides according to methods known in the art); imines of amino containing compounds (e.g. those obtained by condensation with a C₁₋₄ aldehyde or ketone according to methods known in the art); and acetals and ketals of alcohol containing compounds (e.g. those obtained by condensation with chloromethyl methyl ether or chloromethyl ethyl ether according to methods known in the art).

Symptoms of cognitive impairment include difficulty remembering recent events, difficulty remembering recently acquired information, repeating statements, misplacing items, forgetting details of conversations, events, or appointments, not recognizing familiar people and places, having trouble finding the right words to express thoughts or name objects, having difficulty performing calculations, having problems planning and carrying out tasks, such as balancing a checkbook, following a recipe, or writing a letter, having trouble exercising judgment, such as knowing what to do in an emergency, having difficulty controlling moods or behaviors, and not keeping up personal care such as grooming or bathing

It is understood that the list of symptoms of cognitive impairment may be expanded upon in the future as medical science continues to evolve. Thus, the term “symptoms of cognitive impairment” is not to be limited to the list of symptoms provided herein.

As used herein an effective amount of a compound for treating a particular disease is an amount that is sufficient to ameliorate, or in some manner reduce, the symptoms associated with the disease. Such amount may be administered as a single dosage or may be administered according to a regimen, whereby it is effective. The amount may cure the disease but, typically, is administered in order to ameliorate the disease. Typically, repeated administration is required to achieve the desired amelioration of symptoms.

In the general Formula (I), the structural unit having the general Formula (II) may be one or more structural units selected from multiple types of structural units having the general Formula (III).

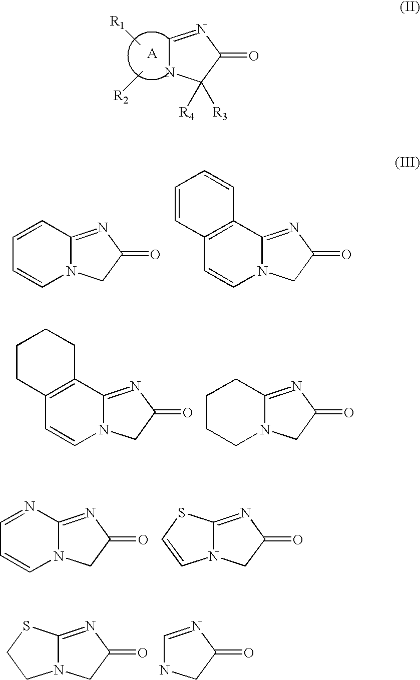

In the general Formula (I), Rₓ is methyl or nil. In the general Formula (I) and Formula (II), R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂-R₅ (wherein R₅ is phenyl (which may be substituted with C₁-C₆ alkyl, halogen atom or cyano) or thienyl) and —O—(CH₂)ₙ-R₆, wherein R₆ is a vinyl group, C₃-C₈ cycloalkyl group, or phenyl group, and n is 0 or 1.

In the general Formula (I) and Formula (II), R₃ and R₄ each are one or more functional groups independently selected from the group consisting of a hydrogen atom, C₁-C₆ alkyl group, C₂-C₆ alkenyl, C₃-C₈ cycloalkyl group, CH₂-R₅ (wherein R₅ is phenyl (which may be substituted with C₁-C₆ alkyl, halogen atom or cyano); naphthyl or thienyl) and —CH(R₈)—R₇. Alternatively, R₃ and R₄ together form a spiro ring having the general Formula (IV):

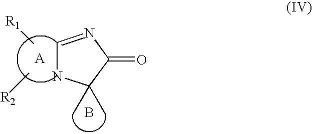

R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group. The above R₈ is a hydrogen atom or C₁-C₆ alkyl group.

Furthermore, in the general Formula (IV), the structural unit B may be one or more structural units selected from multiple types of structural units having the general Formula (V). The structural unit B binds at a position marked by * in the general Formula (V) to form a spiro ring.

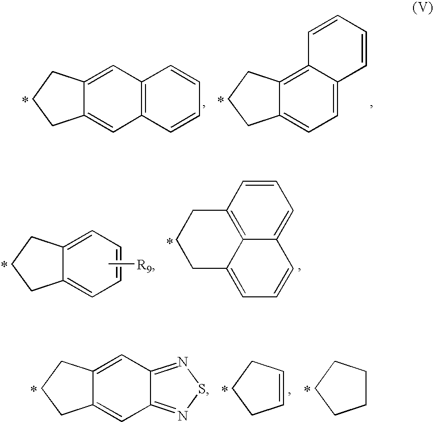

R₉ is one or more functional groups selected from the group consisting of a hydrogen atom, halogen atom, hydroxy group, C₁-C₆ alkoxy group, cyano group, and trifluoromethyl group.

When the heterocyclic compound having the general Formula (I) has asymmetric carbon atoms in the structure, its isomer from asymmetric carbon atoms and their mixture (racemic modification) is present. In such cases, all of them are included in the heterocyclic compound used in the embodiments described later.

The term “C₁-C₆” refers to 1 to 6 carbon atoms unless otherwise defined. The team “C₃-C₈” refers to 3 to 8 carbon atoms unless otherwise defined. The term “C₁-C₆ alkyl” includes linear or branched alkyl groups such as methyl, ethyl, n-propyl, isopropyl, n-butyl, tert-butyl, sec-butyl, n-pentyl, and n-hexyl. The term “C₁-C₆ alkoxy” includes linear or branched alkoxy groups such as methoxy, ethoxy, n-propoxy, isopropoxy, n-butoxy, tert-butoxy, sec-butoxy, n-pentyloxy, and n-hexyloxy. The term “C₃-C₈ cycloalkyl” includes cyclopropyl, cyclobutyl, cyclopentyl, cyclohexyl, cydoheptyl, and cydooctyl. The term “halogen atom” includes fluorine, chlorine, bromine, and iodine.

In another embodiment, the heterocyclic compound useful in the practice of the present invention selected from the group consisting of:
3,3-dimethylimidazo(1,2-a)pyridin-2(3H)-one,
3,3-dipropylimidazo(1,2-a)pyridin-2 (3H)-one,
3,3-dibutylimidazo(1,2-a)pyridin-2(3H)-one,
3,3-diallylimidazo(1,2-a)pyridin-2(3H)-one,
3,3-diallyl-8-benzyloxyimidazo(1,2-a)pyridin-2(3H)-one,
3,3-di(2-propinyl)imidazo(1,2-a)pyridin-2(3H)-one,
3,3-dibenzylimidazo(1,2-a)pyridin-2(3H)-one,
3,3-dibenzyl-8-methylimidazo(1,2-a)pyridin-2(3H)-one,
3,3-dibenzyl-5,7-dimethylimidazo(1,2-a)pyridin-2(3H)-one,
3,3-dibenzyl-8-hydroxyimidazo(1,2-a)pyridin-2(3H)-one,
3,3-dibenzyl-8-methoxyimidazo(1,2-a)pyridin-2(3H)-one,
3,3-dibenzyl-8-ethoxyimidazo(1,2-a)pyridin-2(3H)-one,
8-allyloxy-3,3-dibenzylimidazo(1,2-a)pyridin-2(3H)-one,
3,3-dibenzyl-8-isopropoxyimidazo(1,2-a)pyridin-2(3H)-one,
3,3-dibenzyl-8-cyclopropylmethyloxyimidazo(1,2-a)pyridin-2(3H)-one,
3,3-dibenzyl-8-cycloheptyloxyimidazo(1,2-a)pyridin-2(3H)-one,
3,3-dibenzyl-6-chloroimidazo(1,2-a)pyridin-2(3H)-one,
3,3-dibenzyl-6,8-dichloroimidazo(1,2-a)pyridin-2(3H)-one,
3,3-dibenzyl-8-chloro-6-trifluoromethylimidazo(1,2-a)pyridin-2(3H)-one,
3,3-dibenzyl-8-benzyloxyimidazo(1,2-a)pyridin-2(3H)-one,
8-amino-3,3-dibenzylimidazo(1,2-a)pyridin-2(3H)-one,
8-acetylamino-3,3-dibenzylimidazo(1,2-a)pyridin-2(3H)-one,
3,3-dibenzyl-8-benzylaminoimidazo(1,2-a)pyridin-2(3H)-one,
3,3-bis(3-chlorobenzyl)imidazo(1,2-a)pyridin-2(3H)-one,
3,3-bis(3-fluorobenzyl)imidazo(1,2-a)pyridin-2(3H)-one,
3,3-bis(4-fluorobenzyl)imidazo(1,2-a)pyridin-2(3H)-one,
3,3-bis(2,4-dichlorobenzyl)imidazo(1,2-a)pyridin-2(3H)-one,
3,3-bis(4-dimethylaminobenzyl)imidazo(1,2-a)pyridin-2(3H)-one,
3,3-bis(4-methoxybenzyl)imidazo(1,2-a)pyridin-2(3H)-one,
3,3-bis(4-biphenylmethyl)imidazo(1,2-a)pyridin-2(3H)-one,
3,3-bis(4-cyanobenzyl)imidazo(1,2-a)pyridin-2(3H)-one,
3,3-bis(4-hydroxy-benzyl)imidazo(1,2-a)pyridin-2(3H)-one,
3,3-bis(3-phenyl-1-propyl)imidazo(1,2-a)pyridin-2(3H)-one,
3,3-bis(2,4-difluorobenzyl)imidazo(1,2-a)pyridin-2(3H)-one,
3,3-bis(4-nitrobenzyl)imidazo(1,2-a)pyridin-2(3H)-one,
3,3-bis(4-carboxybenzyl)imidazo(1,2-a)pyridin-2(3H)-one,
8-benzyloxy-3,3-bis(1-phenylethyl)imidazo(1,2-a)pyridin-2(3H)-one,
8-benzyloxy-3,3-bis(3-methylbenzyl)imidazo(1,2-a)pyridin-2(3H)-one,
8-benzyloxy-3,3-bis(4-methylbenzyl)imidazo(1,2-a)pyridin-2(3H)-one,
3-benzyl-3-(4-fluorobenzyl)imidazo(1,2-a)pyridin-2(3H)-one,
3-ethyl-3(4-fluorobenzyl)imidazo(1,2-a)pyridin-2(3H)-one,
8-methyl-3,3-bis(3-pyridylmethyl)imidazo(1,2-a)pyridin-2(3H)-one,
8-methyl-3,3-bis(4-pyridylmethyl)imidazo(1,2-a)pyridin-2(3H)-one,
3,3-bis(2-thienylmethyl)imidazo(1,2-a)pyridin-2(3H)-one,
3,3-bis(2-furylmethyl)imidazo(1,2-a)pyridin-2(3H)-one,
spiro(imidazo(1,2-a)pyridin-2(3H)-one-3,2′-indan),
spiro(imidazo(1,2-a)pyridin-2(3H)-one-3,2′-(2,3)dihydrophenarene),
spiro(imidazo(2,1-b)thiazol-6(5H)-one-5,2′-benzo(f)indan),
spiro(imidazo(1,2-b)thiazol-6(5H)-one-5,2′-indan),
spiro(2-methylimidazo(1,2-b)thiazol-6(5H)-one-5,2′-benzo(f)indan),
5,5-bis(4-fluorobenzyl)imidazo(2,1-b)thiazol-6(5H)-one,
5,5-dibenzylimidazo(2,1-b)thiazol-6(5H)-one,
5,5-bis(4-methylbenzyl)imidazo(2,1-b)thiazol-6(5H)-one,
5,5-bis(4-cyanobenzyl)imidazo(2,1-b)thiazol-6(5H)-one,
5,5-dibenzyl-2-methylimidazo(2,1-b)thiazol-6(5H)-one,
5,5-bis(4-fluorobenzyl)-2-methylimidazo(2,1-b)thiazol-6(5H)-one,
5,5-dicyclohexyl-2-methylimidazo(2,1-b)thiazol-6(5H)-one,
5,5-bis(4-cyanobenzyl)-2-methylimidazo(2,1-b)thiazol-6(5H)-one,
5,5-di(2-butenyl)imidazo(2,1-b)thiazol-6(5H)-one,
5,5-dibutylimidazo(2,1-b)thiazol-6(5H)-one,
5,5-dicyclohexylimidazo(2,1-b)thiazol-6(5H)-one,
5,5-bis(2-thienylmethyl)imidazo(2,1-b)thiazol-6(5H)-one,
spiro(2,3-dihydroimidazo(2,1-b)thiazol-6(5H)-one-5,2′-benzo(f)indan),
5,5-dibutyl-2,3-dihydroimidazo(2,1-b)thiazol-6(5H)-one,
5,5-di(2-butenyl)-2,3-dihydroimidazo(2,1-b)thiazol-6(5H)-one,
5,5-bis(4-methylbenzyl)-2,3-dihydroimidazo(2,1-b)thiazol-6(5H)-one,
5,5-bis(2-thienylmethyl)-2,3-dihydroimidazo(2,1-b)thiazol-6(5H)-one,
5,5-bis(4-fluorobenzyl)-2,3-dihydroimidazo(2,1-b)thiazol-6(5H)-one,
5,5-dibenzyl-2,3-dihydroimidazo(2,1-b)thiazol-6(5H)-one,
spiro(imidazo(1,2-a)pyridin-2(3H)-one-3,2′-benzo(f)indan),
2-hydroxy-3-(2-naphthylmethyl)-imidazo(1,2-a)pyridine,
3-benzylimidazo(1,2-a)pyridin-2(3H)-one,
spiro(5,6,7,8-tetrahydroimidazo(1,2-a)pyridin-2(3H)-one-3,2′-benzo(f)indan),
3,3-dicyclohexyl-5,6,7,8-tetrahydroimidazo(1,2-a)pyridin-2(3H)-one,
3,3-bis(2-thienylmethyl)-5,6,7,8-tetrahydroimidazo(1,2-a)pyridin-2(3H)-one,
3,3-dibutyl-5,6,7,8-tetrahydroimidazo(1,2-a)pyridin-2(3H)-one,
3,3-dipropyl-5,6,7,8-tetrahydroimidazo(1,2-a)pyridin-2(3H)-one,
spiro(imidazo(1,2-a)pyrimidin-2(3H)-one-3,2′-benzo(f)indan),
3,3-di(2-butenyl)imidazo(1,2-a)pyrimidin-2(3H)-one,
3,3-bis(2-thienylmethyl)imidazo(1,2-a)pyrimidin-2(3H)-one,
3,3-bis(4-fluorobenzyl)imidazo(1,2-a)pyrimidin-2(3H)-one,
3,3-dicyclohexylimidazo(1,2-a)pyrimidin-2(3H)-one,
3,3-bis(4-cyanobenzyl)imidazo(1,2-a)pyrimidin-2(3H)-one,
3,3-bis(4-methylbenzyl)imidazo(1,2-a)pyrimidin-2(3H)-one,
4,4-dibenzyl-1-methyl-5-oxo-4,5-dihydroimidazole,
spiro(imidazo(1,2-a)pyridin-2(3H)-one-3,2′-(4′-fluoroindan)),
spiro(imidazo(1,2-a)pyridin-2(3H)-one-3,2′-(5′-methoxyindan)),
spiro(imidazo(1,2-a)pyridin-2(3H)-one-3,2′-(5′-iodoindan)),
spiro(imidazo(1,2-a)pyridin-2(3H)-one-3,2′-(4′-cyanoindan)),
spiro(imidazo(2,1-a)isoquinolin-2(3H)-one-3,2′-indan),
spiro(imidazo(1,2-a)pyridin-2(3H)-one-3,2′-((1,2,5-thiadiazo)(4,5-c)indan)),
spiro(imidazo(2,1-a)isoquinolin-2(3H)-one-3,2′-((1,2,5-thiadiazo)(4,5-c)indan)),
spiro(imidazo(1,2-a)pyrimidin-2(3H)-one-3,4′-(1-cyclopentene)),
spiro(imidazo(1,2-a)pyrimidin-2(3H)-one-3,2′-indan),
spiro(imidazo(1,2-a)pyrimidin-2(3H)-one-3,2′-((1,2,5-thiadiazo)(4,5-c)indan)),
spiro(imidazo(1,2-a)pyridin-2(3H)-one-3,2′-(5′-trifluoromethylindan)),
spiro(imidazo(1,2-a)pyridin-2(3H)-one-3,2′-benzo(e)indan),
spiro(imidazo(2,1-a)isoquinolin-2(3H)-one-3,1′-(3′-cyclopentene)),
spiro(8-benzyloxyimidazo(1,2-a)pyridin-2(3H)-one-3,1′-(3′-cyclopentene)),
spiro(7,8,9,10-tetrahydroimidazo(2,1-a)isoquinolin-2(3H)-one-3,1′-cyclopentane),
spiro(imidazo(2,1-a)isoquinolin-2(3H)-one-3,1′-cyclopentane), and
spiro(5,6,7,8-tetrahydroimidazo(1,2-a)pyridin-2(3H)-one-3,2′-indan).

In another embodiment, the compound is spiro(imidazo(1,2-a)pyridin-2(3H)-one-3,2′-indan).

In another embodiment, the method of the present invention can be practiced using any of the compounds disclosed in U.S. application Ser. No. 11/872,408 (published as US 2008/0103157 A1); U.S. application Ser. No. 11/872,418 (published as US 2008/0103158 A1); U.S. Pat. No. 6,635,652; U.S. Pat. No. 7,141,579; and international Appl. No. PCT/JP2007/070962 (published as WO 2008/047951), each of which is incorporated by reference in its entirety.

The compound ST101, also known as ZSET1446, has shown pharmacological activity in rodent models of learning and memory relevant to AD after both acute (single-dose) and chronic administration. The chemical name for ST101 is spiro(imidazo(1,2-a)pyridin-2(3H)-one-3,2′-indan).

For example, ST101 significantly improves age-impaired memory and attenuates memory deficits induced by chemical amnesic agents such as methamphetamine, the glutamate receptor antagonist, MK-801 and the muscarinic antagonist, scopolamine. (Yamaguchi Y., et al., J. Pharmacol. Exp. Ther. 317:1079-87 (2006); Ito Y., et al., J. Pharmacol. Exp. Ther. 320: 819-27 (2007)).

Experiments have shown that ST101 potentiates nicotine-stimulated release of acetylcholine (ACh), increases extracellular ACh concentrations in the cerebral cortex, and increases extracellular concentrations of both ACh and dopamine in the hippocampus. The breadth of models across which ST101 exerts its effects suggests the potential for involvement at an upstream target in the signaling pathway(s) associated with these processes.

ST101 has also demonstrated effects in the Senescence Accelerated Mouse P8 (SAMP8), a mouse strain that develops age-related deficits in learning and memory along with accumulation of Aβ-like deposits in brain tissue. The SAMP8 mouse is discussed in Morley, J. E., Biogerontology 3: 57-60 (2002). ST101 decreased accumulation of Aβ-like deposits and also produced an improvement in learning and memory functions, suggesting the behavioral effect of ST101 may be linked to reduction of Aβ production and/or deposition. See US 2008/103158 A1.

All patents, patent applications, and publications discussed herein are hereby incorporated by reference in their entireties.

The breadth and scope of the present invention should not be limited by any of the above-described exemplary embodiments, but should be defined only in accordance with the following claims and their equivalents.

What is claimed is:

1. A method of treating or preventing cognitive impairment, said method comprising administering a heterocyclic compound having the general Formula (I): 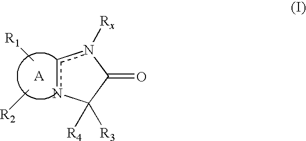
or a pharmaceutically acceptable salt, hydrate or prodrug thereof to a subject in need thereof, wherein
Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂-R₅, and —O—(CH₂)ₙ-R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂-R₅, and —CH(R₈)—R₇; or
(ii) R₃ and R₄ together form a spiro ring of Formula (IV): 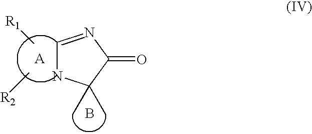
wherein B may be one or more structural units selected from structural units having the general Formula (V), 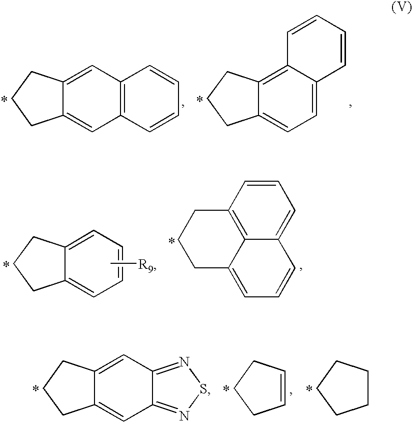
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group;
ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group;
wherein said cognitive impairment is caused by or associated with Huntington's Disease, Lewy Body Dementia, Pick's Disease and Down's Syndrome, and
wherein another agent for treating or preventing neurodegenerative disease is not administered to said subject.

2. The method of claim 1, wherein the heterocyclic compound is spiro(imidazo(1,2-a)pyridin-2(3H)-one-3,2′-indan).

3. The method of claim 1, wherein the cognitive impairment is treated.

4. The method of claim 1, wherein the cognitive impairment is prevented.

5. The method of claim 1, wherein the subject has been diagnosed with Huntington's Disease.

6. The method of claim 1, wherein the subject has been diagnosed as predisposed to Huntington's Disease.

7. The method of claim 1, wherein said subject has been screened to determine whether the subject is predisposed Huntington's Disease.

8. The method of claim 1, wherein the subject has been diagnosed with Lewy Body Dementia.

9. The method of claim 1, wherein the subject has been diagnosed as predisposed to Lewy Body Dementia.

10. The method of claim 1, wherein said subject has been screened to determine whether the subject is predisposed Lewy Body Dementia.

11. The method of claim 1, wherein the subject has been diagnosed with Pick's Disease.

12. The method of claim 1, wherein the subject has been diagnosed as predisposed to Pick's Disease.

13. The method of claim 1, wherein said subject has been screened to determine whether the subject is predisposed Pick's Disease.

14. The method of claim 1, wherein the subject has been diagnosed with Down's Syndrome.

15. The method of claim 1, wherein the subject has been diagnosed as predisposed to Down's Syndrome.

16. The method of claim 1, wherein said subject has been screened to determine whether the subject is predisposed Down's Syndrome.
